SEQUENCE LISTING (50 sequences)

SEQ ID NO: 1 - DNA (genomic), 1431 bp; single-stranded, linear
  units: bp
  authors: Drayna, Dennis Jarnagin, Alisha Stephens McLean, John Henzel, William Kohr, William Fielding, Christopher Lawn, Richard
  title: Cloning and sequencing of human cholesteryl ester transfer protein cDNA
  journal: Nature
  volume: 327
  pages: 632-634
  date: June 18-1987
tgctccaaaggcacctcgcacgaggcaggcatcgtgtgccgcatcaccaagcctgccctcctggtgttgaaccacgagactgccaaggtcatccagaccgccttccagcgagccagctacccagatatcacgggcgagaaggccatgatgctccttggccaagtcaagtatgggttgcacaacatccagatcagccacttgtccatcgccagcagccaggtggagctggtggaagccaagtccattgatgtctccattcagaacgtgtctgtggtcttcaaggggaccctgaagtatggctacaccactgcctggtggctgggtattgatcagtccattgacttcgagatcgactctgccattgacctccagatcaacacacagctgacctgtgactctggtagagtgcggaccgatgcccctgactgctacctgtctttccataagctgctcctgcatctccaaggggagcgagagcctgggtggatcaagcagctgttcacaaatttcatctccttcaccctgaagctggtcctgaagggacagatctgcaaagagatcaacgtcatctctaacatcatggccgattttgtccagacaagggctgccagcatcctttcagatggagacattggggtggacatttccctgacaggtgatcccgtcatcacagcctcctacctggagtcccatcacaagggtcatttcatctacaagaatgtctcagaggacctccccctccccaccttctcgcccacactgctgggggactcccgcatgctgtacttctggttctctgagcgagtcttccactcgctggccaaggtagctttccaggatggccgcctcatgctcagcctgatgggagacgagttcaaggcagtgctggagacctggggcttcaacaccaaccaggaaatcttccaagaggttgtcggcggcttccccagccaggcccaagtcaccgtccactgcctcaagatgcccaagatctcctgccaaaacaagggagtcgtggtcaattcttcagtgatggtgaaattcctctttccacgcccagaccagcaacattctgtagcttacacatttgaagaggatatcgtgactaccgtccaggcctcctattctaagaaaaagctcttcttaagcctcttggatttccagattacaccaaagactgtttccaacttgactgagagcagctccgagtccatccagagcttcctgcagtcaatgatcaccgctgtgggcatccctgaggtcatgtctcggctcgaggtagtgtttacagccctcatgaacagcaaaggcgtgagcctcttcgacatcatcaaccctgagattatcactcgagatggcttcctgctgctgcagatggactttggcttccctgagcacctgctggtggatttcctccagagcttgagctag

SEQ ID NO: 2 - protein, 20 aa
EIFQELSRGLPTGQAQVAVH

SEQ ID NO: 3 - protein, 20 aa
VAVTFRFPRPDGREAVAYRF

SEQ ID NO: 4 - protein, 22 aa
LLLQMDFGFPKHLLVDFLQSLS

SEQ ID NO: 5 - protein, 20 aa
TTVQASYSQKKLFLHLLDFQ

SEQ ID NO: 6 - protein, 20 aa
LLLHLQGEREPGWLKQLFTN

SEQ ID NO: 7 - protein, 20 aa
DVSGERAVMLLGRVKYGLHN

SEQ ID NO: 8 - protein, 20 aa
QEIFQEVVGGFPSQAQVTVH

SEQ ID NO: 9 - protein, 20 aa
VMVKFLFPRPDQQHSVAYTF

SEQ ID NO: 10 - protein, 22 aa
LLLQMDFGFPEHLLVDFLQSLS

SEQ ID NO: 11 - protein, 20 aa
TTVQASYSKKKLFLSLLDFQ

SEQ ID NO: 12 - protein, 20 aa
LLLHLQGEREPGWIKQLFTN

SEQ ID NO: 13 - protein, 20 aa
DITGEKAMMLLGQVKYGLHN

SEQ ID NO: 14 - DNA (genomic), 63 bp; single-stranded, linear
  units: bp
caggaaatcttccaggagctttccagaggccttcccaccggccaggcccaggtagccgtccac

SEQ ID NO: 15 - DNA (genomic), 60 bp; single-stranded, linear
  units: bp
gtcgccgtgacgttccgcttcccccgcccagatggccgagaagctgtggcctacaggttt

SEQ ID NO: 16 - DNA (genomic), 66 bp; single-stranded, linear
  units: bp
ctgctgctgcagatggacttcggttttcccaagcacctgctggtggatttcctgcagagcctgagc

SEQ ID NO: 17 - DNA (genomic), 60 bp; single-stranded, linear
  units: bp
accaccgtccaggcctcctactcccagaaaaagctcttcctacacctcttggatttccag

SEQ ID NO: 18 - DNA (genomic), 60 bp; single-stranded, linear
  units: bp
ctgctcctgcacctccagggggagcgcgagccggggtggctcaagcagctcttcacaaac

SEQ ID NO: 19 - DNA (genomic), 60 bp; single-stranded, linear
  units: bp
gacgtcagcggcgagagggccgtgatgctcctcggccgggtcaagtacgggctgcacaac

SEQ ID NO: 20 - DNA (genomic), 63 bp; single-stranded, linear
  units: bp
caggaaatcttccaagaggttgtcggcggcttccccagccaggcccaagtcaccgtccactgc

SEQ ID NO: 21 - DNA (genomic), 60 bp; single-stranded, linear
  units: bp
gtgatggtgaaattcctctttccacgcccagaccagcaacattctgtagcttacacattt

SEQ ID NO: 22 - DNA (genomic), 66 bp; single-stranded, linear
  units: bp
ctgctgctgcagatggactttggcttccctgagcacctgctggtggatttcctccagagcttgagc

SEQ ID NO: 23 - DNA (genomic), 60 bp; single-stranded, linear
  units: bp
actaccgtccaggcctcctattctaagaaaaagctcttcttaagcctcttggatttccag

SEQ ID NO: 24 - DNA (genomic), 60 bp; single-stranded, linear
  units: bp
ctgctcctgcatctccaaggggagcgagagcctgggtggatcaagcagctgttcacaaat

SEQ ID NO: 25 - DNA (genomic), 60 bp; single-stranded, linear
  units: bp
gatatcacgggcgagaaggccatgatgctccttggccaagtcaagtatgggttgcacaac

SEQ ID NO: 26 - protein, 497 aa
  authors: Nagashima, M. McLean, J. W. Lawn, R. M.
  title: Cloning and mRNA tissue distribution of rabbit cholesteryl ester transfer protein
  journal: J. Lipid Res.
  volume: 29
  pages: 1643-1649
  date: 1988
ACPKGASYEAGIVCRITKPALLVLNQETAKVVQTAFQRAGYPDVSGERAVMLLGRVKYGLHNLQISHLSIASSQVELVDAKTIDVAIQNVSVVFKGTLNYSYTSAWGLGINQSVDFEIDSAIDLQINTELTCDAGSVRTNAPDCYLAFHKLLLHLQGEREPGWLKQLFTNFISFTLKLILKRQVCNEINTISNIMADFVQTRAASILSDGDIGVDISVTGAPVITATYLESHHKGHFTHKNVSEAFPLRAFPPGLLGDSRMLYFWFSDQVLNSLARAAFQEGRLVLSLTGDEFKKVLETQGFDTNQEIFQELSRGLPTGQAQVAVHCLKVPKISCQNRGVVVSSSVAVTFRFPRPDGREAVAYRFEEDIITTVQASYSQKKLFLHLLDFQCVPASGRAGSSANLSVALRTEAKAVSNLTESRSESLQSSLRSLIATVGIPEVMSRLEVAFTALMNSKGLDLFEIINPEIITLDGCLLLQMDFGFPKHLLVDFLQSLS

SEQ ID NO: 27 - DNA (genomic), 1494 bp; single-stranded, linear
  units: bp
  authors: Nagashima, Mariko McLean, John W. Lawn, Richard M.
  title: Cloning and mRNA tissue distribution of rabbit cholesteryl ester transfer protein
  journal: J. Lipid Res.
  volume: 29
  pages: 1643-1649
  date: 1988
gcctgtcccaaaggcgcctcctacgaggctggcatcgtgtgtcgcatcaccaagcccgccctcttggtgttgaaccaagagacggccaaggtggtccagacggccttccagcgcgccggctatccggacgtcagcggcgagagggccgtgatgctcctcggccgggtcaagtacgggctgcacaacctccagatcagccacctgtccatcgccagcagccaggtggagctggtggacgccaagaccatcgacgtcgccatccagaacgtgtccgtggtcttcaaggggaccctgaactacagctacacgagtgcctgggggttgggcatcaatcagtctgtcgacttcgagatcgactctgccattgacctccagatcaacacagagctgacctgcgacgctggcagtgtgcgcaccaatgcccccgactgctacctggctttccataaactgctcctgcacctccagggggagcgcgagccggggtggctcaagcagctcttcacaaacttcatctccttcaccctgaagctgattctgaagcgacaggtctgcaatgagatcaacaccatctccaacatcatggctgactttgtccagacgagggccgccagcatcctctcagatggagacatcggggtggacatttccgtgacgggggcccctgtcatcacagccacctacctggagtcccatcacaagggtcacttcacgcacaagaacgtctccgaggccttccccctccgcgccttcccgcccggtcttctgggggactcccgcatgctctacttctggttctccgatcaagtgctcaactccctggccagggccgccttccaggagggccgtctcgtgctcagcctgacaggggatgagttcaagaaagtgctggagacccagggtttcgacaccaaccaggaaatcttccaggagctttccagaggccttcccaccggccaggcccaggtagccgtccactgccttaaggtgcccaagatctcctgccagaaccggggtgtcgtggtgtcttcttccgtcgccgtgacgttccgcttcccccgcccagatggccgagaagctgtggcctacaggtttgaggaggatatcatcaccaccgtccaggcctcctactcccagaaaaagctcttcctacacctcttggatttccagtgcgtgccggccagcggaagggcaggcagctcagcaaatctctccgtggccctcaggactgaggctaaggctgtttccaacctgactgagagccgctccgagtccctgcagagctctctccgctccctgatcgccacggtgggcatcccggaggtcatgtctcggctcgaggtggcgttcacagccctcatgaacagcaaaggcctggacctcttcgaaatcatcaaccccgagattatcactctcgatggctgcctgctgctgcagatggacttcggttttcccaagcacctgctggtggatttcctgcagagcctgagctag

SEQ ID NO: 28 - protein, 476 aa
  authors: Drayna, Dennis Jarnagin, Alisha Stephens McLean, John Henzel, William Kohr, William Fielding, Christopher Lawn, Richard
  title: Cloning and sequencing of human cholesteryl ester transfer protein cDNA
  journal: Nature
  volume: 327
  pages: 632-634
  date: June 18-1987
CSKGTSHEAGIVCRITKPALLVLNHETAKVIQTAFQRASYPDITGEKAMMLLGQVKYGLHNIQISHLSIASSQVELVEAKSIDVSIQNVSVVFKGTLKYGYTTAWWLGIDQSIDFEIDSAIDLQINTQLTCDSGRVRTDAPDCYLSFHKLLLHLQGEREPGWIKQLFTNFISFTLKLVLKGQICKEINVISNIMADFVQTRAASILSDGDIGVDISLTGDPVITASYLESHHKGHFIYKNVSEDLPLPTFSPTLLGDSRMLYFWFSERVFHSLAKVAFQDGRLMLSLMGDEFKAVLETWGFNTNQEIFQEVVGGFPSQAQVTVHCLKMPKISCQNKGVVVNSSVMVKFLFPRPDQQHSVAYYFEEDIVTTVQASYSKKKLFLSLLDFQITPKTVSNLTESSSESIQSFLQSMITAVGIPEVMSRLEVVFTALMNSKGVSLFDIINPEIITRDGFLLLQMDFGFPEHLLVDFLQSLS

SEQ ID NO: 29 - protein, 26 aa
  authors: Swenson, T. L. et al.,
  journal: J. Biol. Chem.
  volume: 264
  pages: 14318-14326
  date: 1989
RDGFLLLQMDFGFPEHLLVDFLQSLS

SEQ ID NO: 30 - protein, 493 aa
  authors: Pape, Michael E. Rehberg, Edward F. Marotti, Keith R. Melchior, George W.
  title: Molecular Cloning, Sequence, and Expression of Cynomolgus Monkey Cholesteryl Ester Transfer Protein
  journal: Arteriosclerosis and Thrombosis
  volume: 11
  issue: 6
  pages: 1759-1771
  date: Nov/Dec-1991
MLAATVLTLALLGNVHACSKGTSHKAGIVCRITKPALLVLNQETAKVIQSAFQRANYPNITGEKAMMLLGQVKYGLHNIQISHLSIASSRVELVEAKSIDVSIQNVSVVFKGTLKYGYTTAWGLGIDQSVDFEIDSAIDLQINTQLTCDSGRVRTDAPDCYLSFHKLLLHLQGEREPGWIKQLFTNFISFTLKLVLKGQICKEINIISNIMADFVQTRAASILSDGDIGVDISLTGDPIITASYLESHHKGYFIYKNVSEDLPLPTFSPALLGDSRMLYFWFSEQVFHSLAKVAFQDGRLTLSLMGDEFKAVLETWGFNTNQEIFQEVVGGFPSQAQVTVHCLKMPRISCQNKGVVVNSSVMVKFLFPRPDQQHSVAYTFEEDIMTTVQASYSKKKLFLSLLDFQITPKTVSNLTESSSESVQSFLQSMITTVGIPEVMSRLEAVFTALMNSKGLSLFDIINPEIITRDGFLLLQMDFGFPEHLLVDFLQSLS

SEQ ID NO: 31 - DNA (genomic), 1508 bp; single-stranded, linear
  authors: Pape, Michael E. Rehberg, Edward F. Marotti, Keith R. Melchior, George W.
  title: Molecular Cloning, Sequence, and Expression of Cynomolgus Monkey Cholesteryl Ester Transfer Protein
  journal: Arteriosclerosis and Thrombosis
  volume: 11
  issue: 6
  pages: 1759-1771
  date: Nov/Dec-1991
atgctggctgccaccgtcctgaccctggccctgctgggcaatgtccacgcctgctccaaaggtacctcacacaaggcaggcattgtgtgccgcatcaccaagcctgccctcctggtgttgaaccaacagactgccaaggtgatccagtctgccttccagcgagccaactacccaaatatcacaggcgagaaggccatgatgctccttggccaagtcaagtatgggttgcacaacatccaaatcagccacttgtccatcgccagcagccgggtggagctggtggaagccaagtccattgatgtctccattcagaacgtgtctgtggtcttcaaggggaccctgaagtatggctacaccactgcctgggggctgggcattgatcagtccgttgacttcgagatcgactctgccattgacctccagatcaacacacaactgacctgtgactctggtagagtgaggactgatgcccctgactgctacctgtctttccataagctgctcctgcatctccaaggggagcgagagcccgggtggatcaagcagctgttcacaaacttcatctccttcaccctgaagctggtcctgaagggacagatctgcaaagagatcaacatcatctccaacatcatggccgattttgtccagacaagggctgccagtatcctttcagatggagacatcggggtggacatttccctgacaggtgatcccatcattacagcctcctacctggagtcccatcacaagggttatttcatctataagaatgtctcggaggacctcccactccccaccttctcgcccgcactgctgggggactcccgcatgctgtacttctggttctccgagcaagtcttccactccctggccaaggtagctttccaagatgcccgcctcacgctcagcctgatgggagacgagttcaaggcagtgctggagacctggggcttcaacaccaaccaagaaatcttccaggaggttgtcggcggcttccccagccaggcccaagtcaccgtccactgcctcaagatgcccaggatctcctgccaaaacaagggagtcgtggtcaattcttcggtgatggtgaaattcctctttccacgcccagaccagcaacactctgtagcttacacatttgaagaggatatcatgaccaccgtccaggcctcctattctaagaaaaagctcttcttaagcctcttggatttccagattacaccaaagactgtttccaacttgactgagagcagctccgagtccgtccagagcttcctgcagtcaatgatcaccactgtgggcatccctgaggtcatgtctcggcttgaggcagtgtttacagccctcatgaacagcaaaggcctgagcctcttcgacatcatcaatcctgagattatcactcgagatggcttcctgctgctgcagatggactttggcttccctgagcacctgctggtggatttcctccagagcttgagctagaagtctccaaggacgtcaggatgggg

SEQ ID NO: 32 - protein, 20 aa
QEIFQEVVGGFPSQAQVTVH

SEQ ID NO: 33 - protein, 20 aa
VMVKFLFPRPDQQHSVAYTF

SEQ ID NO: 34 - protein, 22 aa
LLLQMDFGFPEHLLVDFLQSLS

SEQ ID NO: 35 - protein, 20 aa
TTVQASYSKKKLFLSLLDFQ

SEQ ID NO: 36 - protein, 20 aa
LLLHLQGEREPGWIKQLFTN

SEQ ID NO: 37 - protein, 20 aa
NITGEKAMMLLGQVKYGLHN

SEQ ID NO: 38 - protein, 183 aa
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 39 - DNA (genomic), 552 bp; single-stranded, linear
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttag

SEQ ID NO: 40 - protein, 25 aa
TWVGVNLEDPASRDLVVSYVNTNMG

SEQ ID NO: 41 - protein, 21 aa
LLWFHISCLTFGRETVIEYLV

SEQ ID NO: 42 - protein, 16 aa
VVSYVNTNMGLKFRQL

SEQ ID NO: 43 - protein, 21 aa
VSFGVWIRTPPAYRPPNAPIL

SEQ ID NO: 44 - DNA (cDNA), 35 bp; single-stranded, linear
gatcccatggacatcgacccttataaagaatttgg

SEQ ID NO: 45 - DNA (cDNA), 44 bp; single-stranded, linear
gatcaagcttttaacattgagattcccgagattgagatcttctg

SEQ ID NO: 46 - DNA (cDNA), 43 bp; single-stranded, linear
gatcgaattcactagttggaagatccagcgtctagagacctag

SEQ ID NO: 47 - DNA (cDNA), 41 bp; single-stranded, linear
gatcgaattcctcgagctagagtcattagttccccccagca

SEQ ID NO: 48 - DNA (cDNA), 34 bp; single-stranded, linear
gattatcactcgagatggcttcctgctgctgcag

SEQ ID NO: 49 - DNA (cDNA), 40 bp; single-stranded, linear
gatcgaattcagcgctcaagctctggaggaaatccaccag

SEQ ID NO: 50 - protein, 26 aa
LDGCLLLQMDFGFPKHLLVDFLQSLS

(19) United States
(12) Patent Application Publication
(10) Pub. No.: US 2003/0021804 A1
(43) Pub. Date: Jan. 30, 2003

(54) IMMUNOLOGICAL PROCESS FOR INCREASING THE HDL CHOLESTROL CONCENTRATION

(76) Inventors: PHILIP NEEDLEMAN, CREVE COEUR, MO (US); KEVIN GLENN, MARYLAND HEIGHTS, MO (US)

Correspondence Address: WELSH & KATZ LTD, 22ND FLOOR, 120 SOUTH RIVERSIDE PLAZA, CHICAGO, IL, 60606

(21) Appl. No.: 08/785,997

(22) Filed: Jan. 21, 1997

(51) Int. Cl. A61K 39/00; A61K 39/38; A61K 39/385; C07K 5/00; C07K 7/00; C07K 16/00; C07K 17/00; A61K 38/00; A61K 38/04

(52) U.S. Cl. 424/193.1; 424/184.1; 424/185.1; 424/192.1; 530/324; 530/326; 530/327

(57) ABSTRACT

A process for increasing the concentration of HDL cholesterol in the blood of a mammal whose blood contains cholesterol ester transfer protein (CETP) is contemplated. That process comprises the steps of: (a) immunizing the mammal with an inoculum containing a CETP immunogen that is an immunogenic polypeptide having a CETP amino acid residue sequence and is dissolved or dispersed in a vehicle; and (b) maintaining the immunized mammal for a time period sufficient for said immunogenic polypeptide to induce the production of antibodies that bind to CETP and lessen the transfer of cholesteryl esters from HDL. Immunogens, inocula and DNA segments useful for carrying out the invention are also disclosed.

### TECHNICAL FIELD

The present invention relates to a process for for raising the HDL cholesterol level in mammalian blood, and more particularly to an immunological process for ameliorating dyslipoproteinemias characterized by low HDL/LDL cholesterol ratios.

### BACKGROUND OF THE INVENTION

Cholesteryl ester transfer protein (CETP) is an acidic plasma glycoprotein that plays a critical role in establishing high density lipoprotein (HDL), low density lipoprotein (LDL), and very low density lipoprotein (VLDL) cholesterol blood plasma levels and lipid composition in plasma [L. Lagrost, Biochem. Biophys. Acta., 1215:209-236 (1994)]. Several studies, some of which are discussed below, have demonstrated that CETP mediates the transfer of cholesterol esters (CE) from HDL particles to LDL and VLDL particles, as well as mediating the transfer of triglycerides (TG) from LDL and VLDL to HDL particles. This reciprocal exchange of CE and TG is the primary means of providing CE to LDL and VLDL particles in many mammalian species. CETP thus mediates the balanced exchange of cholesteryl esters (CE) and triglycerides (TG) between pro-atherogenic (LDL and VLDL) and anti-atherogenic (HDL) lipoprotein fractions.

Mammalian species whose blood plasma contains CETP such as humans and other primates, rabbits, and hamsters suffer atherosclerosis and heart disease when exposed to diets rich in cholesterol. Other animal species such as mice, rats and dogs lack plasma CETP (measured as transfer activity) and are not susceptible to dietary cholesterol-induced atherosclerosis.

That CETP contributes to the pathogenesis of atherosclerosis in humans has been strongly supported by transgenic mouse studies [G. Melchior et al., Trends in Card. Med, 5:83-87 (1995)]. For example, transgenic mice having a mini gene of cynomolgus monkey CETP cDNA plus the proximal region of the CETP promoter show dietary cholesterol regulation of CETP levels similar to those seen in humans, hamsters and monkeys. Those transgenic mice expressing high levels of the monkey CETP (levels comparable to human dyslipidemias) exhibit: increased LDL+VLDL cholesterol and apo-B and, decreased HDL cholesterol, LDL-receptor and HMG-COA reductase mRNA. Atheroma could be induced by high fat diet in transgenic mice with the cynomolgus monkey CETP transgene.

The CETP amino acid residue and nucleotide sequences of mammalian species have been characterized. For example, the human CETP DNA sequence of SEQ ID NO:1 has been determined [D. Drayna et al., Nature, 327:632-634 (1987)]. The rabbit CETP DNA sequence of SEQ ID NO:27 has also been characterized [M. Nagashima et al., J. Lipid Res., 29:1643-1469 (1988)], as has the cynomolgus monkey CETP sequence [M. E. Pape et al., Atherosclerosis and Thrombosis, 11:1759-1771 (1991)]. The human CETP protein is 476 amino acid residues long, whereas the rabbit CETP protein is 496 amino acid residues long, and the cynomolgus monkey sequence contains 476 residues.

CETP may be a key factor for the global regulation of atherogenicity of plasma lipoproteins in patients with atherosclerosis or coronary artery disease (CAD). CAD is the number one cause of morbidity and mortality in western society. Patients at increased risk for developing coronary artery disease typically exhibit an enhanced level of CETP activity. It has also been reported that CETP has higher affinity for oxidized LDL than native LDL molecules [L. Lagrost, Biochem. Biophys. Acta., 1215:209-236 (1994)]. High levels of LDL cholesterol (>180 mg/dl) [J. Am. Med. Assoc., 269:3015-3023 (1993) and A. L. Gould et al., Circulation, 91:2274-2282 (1995)]; and low levels of HDL cholesterol (<35 mg/dl) [G. Assman et al., Excerpta Medica, 46-59 (1989) and V. Manninen et al., Circulation, 85:37-45 (1992)] have been reported to be important contributors to the development of atherosclerosis.

Individuals who possess genetic deficiencies of the CETP protein have elevated HDL cholesterol levels. Heterozygotes have HDL levels 15-20 percent above non-affected controls. It has been suggested that there is a 2-3 percent decrease in incidence for CAD for each 1 mg/dl increase in HDL cholesterol after correction for other risk factors [D. J. Grodon et al., Nature, 79:8-15, (1989)].

In an experimental model of CETP deficiency in hamsters, it has been shown that passive transfer of mouse anti-human CETP monoclonal antibodies (1C4) inhibited hamster plasma CETP CE transfer by 70-80 percent at all times up to 24 hours following injection of 500 μg of 1C4 (approximately 3.7 mg/kg body weight). That inhibition of hamster CETP-mediated transfer in vivo increased hamster HDL cholesterol by 33 percent, increased HDL-CE by 31 percent and decreased HDL-TG by 42 percent. These results indicate an example of mammalian (hamster) CETP-mediated CE-TG exchange being disrupted by xenogeneic anti-human CETP monoclonal antibodies, and further demonstrate the use of hamsters as pre-clinical model for testing CETP inhibition [B. J. Gaynor et al., Atherosclerosis, 110(1):101-109 (1994)].

In another study reported by G. W. Melchior et al., J. Biol. Chem., 270(36):21068-74 (1995) cynomolgus monkey CETP was shown to have two neutral lipid binding sites. A monoclonal antibody to purified cynomolgus monkey CETP identified as CMTP-2 was capable of severely inhibiting triglyceride (TG) transfer, but had a variable effect on cholesteryl ester (CE) transfer.

Thus, when the monoclonal antibody was administered sub-cutaneously to cynomolgus monkeys at a dose that inhibited TG transfer in the plasma by more than 90 percent, there was no detectable effect on the high density lipoprotein cholesterol level, but the HDL-TG levels decreased from 13 to 1 mol/mol of HDL. A Fab antibody fragment had no effect on CE transfer, but completely blocked TG transfer. Another type of inhibitor, 6-chloromercuric cholesterol, severely inhibited CE transfer with minimal inhibition of TG transfer. When both the inhibitory monoclonal antibody and the 6-chloromercuric cholesterol were added to the assay, both CE and TG transfer were inhibited, indicating that the inhibitors did not compete for the same binding site on CETP. This study indicated that in vivo administration of xenogeneic monoclonal antibodies uncoupled CE and TG transfer.

The inhibitory effects of antisense RNA on expression of CETP protein were reported using vaccinia virus as an expression system. [M. H. Lee et al., J. Biochem. Mol. Biol., 28(3):243-248 (1995)]. The cDNA from CETP was inserted into a transfer vector (pSC11) in sense and antisense orientations and then used to construct recombinant vaccinia viruses. Decreased expression of the exogenous CETP cDNA in mouse cells was clearly evident in the Northern and Western blot analyses as the dose of anti-sense expression increased. Also, in the CETP assay, the CETP activity was decreased compared to the activity obtained from the cell extracts infected with sense constructs only.

More recently, Sugano et al., J. Biol. Chem., 271(32):19080-19083 (1996) reported upon the in vivo effects of antisense CETP RNA administration to rabbits. In that report, a decrease in total cholesterol and CETP activity levels were found 24, 48 and 96 hours following antisense CETP administration, as was an increase in plasma HDL cholesterol at 48 hours.

Other methods of inhibition of CETP-mediated transfer are described in the literature. For example, data from Parke-Davis company has shown that infusion of 10 to 20 mpk of the small molecule compound referred to as PD 140195 into rabbits inhibited CETP activity within 30 minutes (measured in an ex vivo assay) [C. Bisgaier et al., Lipids, 811-818 (1994)]. Schering-Plough Company has published on the isolation of Wiedendiol-A and -B from a marine sponge and has shown that this class of compounds to be low μM inhibitors of CETP-mediated CE transfer in vitro [S. Coval et al., Bioorganic & Med. claim. Lett., 5:605-619 (1995)].

Currently, nicotinic acid and the fibrate drugs are the only small molecule drug therapies that cause significant rises in HDL cholesterol. These drugs are poorly tolerated and must be taken daily. Therapeutic doses of these drugs lead to 15-20 percent increases in HDL cholesterol.

Three mouse monoclonal antibodies to human CETP that recognize a similar epitope on CETP, caused parallel and complete in vitro immunotitration of human plasma CE and triglyceride transfer activities, but only partial inhibition of phospholipid transfer activity [C. B. Hesler et al., J. Biol. Chem., 263(11):5020-5023 (1988)]. Those three monoclonals were originally designated 5C7, 2H4 and 7E1, but in more recent publications of the authors, those monoclonals are referred to as TP2, TP1 and TP3, respectively.

Monoclonal antibody TP2 is directed against an epitope within the last 26 amino acids of CETP (SEQ ID NO:29) [T. L. Swenson et al., J. Biol. Chem., 264:14318-14326 (1989)], and more particularly to an epitope between about positions 465 and 475 of SEQ ID NO:28 [Tall, J. Lipid Res., 34:1255-1274 (1993)]. That monoclonal has been shown to block CETP-mediated lipid transfer by limiting access to lipid-binding sites in the carboxy-terminus of CETP.

In an in vivo study using the xenogeneic mouse monoclonal antibodies (TP1) to CETP, rabbits were intravenously injected with TP1, or irrelevant monoclonal antibodies or saline (control), resulting in an initial 70 percent inhibition of CETP-mediated CE transfer activity. Inhibition was 45 percent after 48 hours for the TP1-injected animals. HDL-CE increased in TP1-treated animals and reached levels that doubled over initial and control values at 48 hours. HDL-TG fell reciprocally, but HDL protein did not change, suggesting a CE for TG exchange. VLDL CE-TG ratio also decreased. CETP inhibition delayed the initial clearance of radioactively-tagged HDL, suggesting that CETP plays a quantitative role in HDL-CE catabolism in the rabbit, promoting the exchange of TG for CE, and the clearance of CE from plasma [M. E. Whitlock et al., J. Clin. Invest., 84:129-137 (1989)].

In further animal studies with hamsters, a single sub-cutaneous injection of TP2 monoclonal antibodies in another illustration of passive administration of xenogeneic antibodies decreased CETP-mediated activity by 58 percent, lowered LDL+VLDL cholesterol 32 percent and raised HDL cholesterol 24 percent [G. Evans et al., J. Lipid Res. 35:1634-1645 (1994) and S. Zuckerman et al., Lipids, 30:307-311 (1995)]. The effect of the TP2 monoclonal antibodies on CETP-mediated CE transfer inhibition was evident within 24 hours after injection and was maximized by 4 days. Lipoproteins returned to control levels 14 days after TP2 administration. The shift in the ratio of VLDL+LDL cholesterol to HDL cholesterol levels due to TP2 monoclonal antibody administration was more significant in hypercholesterolemic hamsters.

TP2 also has a higher efficacy in hamsters fed with a western diet enriched in cholesterol. CETP-mediated activity was reportedly increased in these animals 2-fold over chow-fed hamsters.

The preparation of recombinant CETP molecules has been reported by several research groups. For example, in a study reported recently, glutathione S-transferase-human CETP fusion protein (86 kDa) was expressed using vaccinia viral transfer vectors transfected into CV-1 monkey kidney cells. Using a Western blot assay, the fusion protein was identified by polyclonal antibodies against the carboxy-terminal active region of CETP fused with GST. After cleavage of the GST portion of the fusion protein, the purified CETP showed biological activity in a CETP in vitro assay [W. H. Yoon et al., Mol. Cells, 5(2):107-113 (1995)] and M. K. Jang et al., J. Biochem. Mol. Biol., 28(3):216-220 (1995)].

It has also been reported that specific rabbit polyclonal antibodies were produced by immunization with a GST-CETP fusion protein. A full-length CETP cDNA clone isolated from a human heart λgt11 library was used to provide the C-terminal 94 bp of CETP after a full length CETP molecule expressed in E. coli was found to be insoluble. The lambda gt11 cDNA library was subcloned into pGEX plasmid and a GST-CETP fusion protein was expressed in E. coli. The CETP-GST fusion protein was purified by glutathione-Sepharose-4B affinity chromatography and used as an antigen for the production of rabbit polyclonal antibodies. The antibodies showed good titers, not only against the GST-CETP fusion protein, but also against a mixture of synthetic peptides corresponding in sequence to two 16-mers from the carboxy-terminal region of human CETP. The antibodies were said to be useful as an immunological tool for a CETP assay [N. W. Jeong et al., Mol. Cells, 4(4):529-533 (1994)].

To date there are no published reports on the long-term inhibition of CETP-mediated CE transfer. Passive immunization with the use of xenogeneic antibodies can only be utilized for a short-term period of time because host animals develop antibodies to the xenogeneic immunoglobulin. The invention described hereinafter provides an autogeneic immunological means for the long-term lessening of transfer of cholesteryl esters from HDL particles in mammals whose blood contains CETP. This permits the long-term elevation of anti-atherogenic HDL cholesterol concentrations.

### BRIEF SUMMARY OF THE INVENTION

The present invention contemplates an autogeneic immunological process for lessening the transfer of cholesteryl esters from HDL particles and for increasing the HDL cholesterol concentration of a mammal whose blood also contains CETP. A contemplated process is useful in treating human pro-atherogenic dyslipoproteinemias characterized by low HDL/LDL cholesterol ratios. Also contemplated here are immunogens utilized in that process, as well as isolated and purified DNA that encodes some of those immunogens and expression systems for that DNA.

One contemplated process comprises the steps of:

(a) immunizing the mammal to be treated with an inoculum containing a CETP immunogen that is an immunogenic polypeptide having a CETP amino acid residue sequence and is dissolved or dispersed in a vehicle; and

(b) maintaining the immunized mammal for a time period sufficient for the immunogenic polypeptide to induce the production of antibodies that bind to CETP and lessen the transfer of cholesteryl esters from HDL. In one embodiment, the immunogenic polypeptide is an intact CETP molecule such as recombinant human or rabbit CETP. In another embodiment, the immunogenic polypeptide is a portion of a CETP molecule that is covalently bonded to an exogenous antigenic carrier.

In preferred embodiments, the exogenous antigenic carrier is the hepatitis B core protein (HBcAg), tetanus toxoid, tuberculin purified protein derivative (PPD), diphtheria toxoid or branched oligolysine. HBcAg is particularly preferred as an exogenous antigenic carrier, and is more preferred when utilized as a fusion protein with the immunogenic polypeptide having an amino acid residue sequence of the carboxy-terminal 30 residues of CETP. That more preferred fusion protein constitutes a polypeptide having the amino acid residue sequence of the hepatitis B core antigen from which about 3 to about 53 amino acid residues have been deleted and replaced by the immunogenic polypeptide that more preferably still has a length about equal to the number of amino acid residues deleted from HBcAg. The resulting fusion protein is most preferably present in the inoculum as particles having the size of HBcAg particles (about 27 nm).

The present invention has several benefits and advantages. One salient benefit is that a contemplated process can be utilized to lessen the CE transfer from HDL to LDL or VLDL, thereby increasing the concentration of anti-atherogenic HDL cholesterol.

An advantage of the invention is that a contemplated process can have an effect that lasts for months as compared to the short-term effects of the small molecule drugs non available.

Another benefit of a contemplated process is that it utilizes the host mammal's own (autogeneic) immunological system to provide a desired result, thereby obviating problems associated with repeated administration of xenogeneic antibodies that themselves become immunogenic in the host mammal.

Another advantage of some contemplated processes is that their use of well known and accepted exogenous antigenic carriers such as HBcAg, tetanus toxoid, tuberculin PPD and diphtheria toxoid can boost the host mammal's immunity to those pathogens.

Still further benefits and advantages of the present invention will become apparent to a skilled worker from the disclosure that follows.

### DEFINITIONS

The term “recombinant” is used to denote a version of a molecule made by a process by which a gene is cloned and expressed using recombinant DNA technology and genetic engineering in a bacterial, viral or mammalian host cells to produce an expressed protein in a recombinant form.

The term “polypeptide” is used herein to denote a sequence of about 10 to about 500 peptide-bonded amino acid residues. A whole protein as well as a portion of a protein having the stated minimal length are polypeptides.

The term “whole length CETP” is used to denote the full length CETP molecule (for example 476 amino acid residues long for human CETP or 496 residues long for rabbit CETP) as available in nature or produced as a recombinant protein.

The term “CETP immunogen” is used to denote molecule that is used to induce the production of antibodies that immunoreact with (bind to) CETP.

The terms “immunogenic polypeptide having a CETP amino acid residue sequence” or “immunogenic polypeptide” are used to denote the anti-CETP antibody-inducing portion of a “CETP immunogen”; i.e., that portion of a CETP immunogen to which induced antibodies bind.

The term “fusion protein” is used to denote the expression product of two or more different genes in which the amino acid residue sequences of both genes are expressed peptide-bonded together as a single molecule. It is noted that a fusion protein need not have the full length amino acid residue sequence of any protein, but rather usually contains two or more truncated sequences. The term is therefore somewhat of a misnomer, but is nonetheless well known and used as defined here by those skilled in the art.

The terms “exogenous antigenic carrier” or “carrier” is used herein to denote a molecule foreign to the immunized mammal that provides a signal to antibody-producing B cells. Such carriers and their functions are well known in the art. Such a carrier can be a polypeptide having a sequence of as few as about 10 amino acid residues to the length of an intact protein, as well as being a synthetic polymer or oligomer.

The term “inoculum” in its various grammatical forms is used herein to describe a composition containing an amount of CETP immunogen (e.g., polypeptide conjugate, CETP protein or recombinant protein) sufficient for a described purpose that is dissolved or dispersed in an aqueous, physiologically tolerable diluent.

The term “expression” is used to mean the combination of intracellular processes, including transcription and translation undergone by a structural gene to produce a polypeptide.

The terms “operatively linked” or “operably inserted” are used to mean that two or more DNA sequences are covalently bonded together in correct reading frame.

The term “promoter” is used to mean a recognition site on a DNA sequence or group of DNA sequences that provide an expression control element for a gene and to which RNA polymerase specifically binds and initiates RNA synthesis (transcription) of that gene.

The term “recombinant DNA molecule” is used to mean a hybrid DNA sequence comprising at least two nucleotide sequences not normally found together in nature.

The term “structural gene” is used to mean a DNA sequence that is expressed as a polypeptide; i.e., an amino acid residue sequence.

The term “vector” is used to mean DNA molecule capable of replication in a cell and/or to which another DNA segment can be operatively linked so as to bring about replication of the attached segment. A plasmid is an exemplary vector.

The term “expression vector” is used to mean a DNA sequence that forms control elements that regulate expression of structural genes when operatively linked to those genes within a vector.

### DETAILED DESCRIPTION OF THE INVENTION

The present invention relates to a process for increasing the ratio of HDL cholesterol to LDL cholesterol in the blood of a treated mammal that has CETP in its blood, and that in humans leads to an amelioration of dyslipoproteinemias characterized by low HDL/LDL cholesterol ratios. That desired raising of the HDL/LDL cholesterol ratio is accomplished immunologically by antibodies induced in the blood of the treated mammal that recognize circulating CETP. Also contemplated in this invention are an immunogen utilized in the process, an inoculum that utilizes the immunogen and an isolated and purified DNA segment that encodes a contemplated immunogen.

I. The Process

A contemplated process is referred to herein as utilizing “autogeneic” antibodies to denote that the useful antibodies are those induced in the host mammal itself. This autogeneic immunological process is therefore to be distinguished from a xenogeneic process in which antibodies from an animal of one species are administered to an animal of another species as where the mouse anti-CETP TP2 or 1C4 monoclonal antibodies have been administered to hamsters or rabbits. A contemplated autogeneic immunological process is also to be distinguished from an allogeneic immunological process such as a passive immunization in which antibodies from one animal are administered to another animal of the same species as where humans receive gamma globulin injections from other humans.

A contemplated process is thus closely analogous to an autoimmune process in which a mammal's own immune system attacks an endogenous or self protein. CETP is an endogenous protein in rabbits, hamsters and primates that are among the mammalian hosts contemplated here. However, inasmuch as the cause of most if not all autoimmune responses is presently unknown and the desired immune response contemplated here is purposefully induced, it is believed appropriate to use a different name for the result obtained here.

Thus, one aspect of the present invention contemplates a process for lessening the transfer of cholesteryl esters from HDL particles and increasing the concentration of HDL cholesterol in the blood of a mammal whose blood contains cholesterol ester transfer protein (CETP). That process comprises the steps of: (a) immunizing that mammal (the host) with an inoculum that contains a CETP immunogen dissolved or dispersed in a vehicle. The CETP immunogen is an immunogenic polypeptide having a CETP amino acid residue sequence. The immunized mammal is (b) maintained for a time period sufficient for the immunogenic polypeptide to induce the production of antibodies that bind to CETP and lessen the transfer of cholesteryl esters (CE) from HDL.

A. The CETP Immunogen

The immunogenic polypeptide having a CETP amino acid residue sequence of the CETP immunogen can be a whole CETP molecule such as the human (476 residues) or rabbit (496 residues) proteins whose amino acid residue sequences are provided as SEQ ID NOs:28 and 26, respectively, and whose DNA sequences are provided in SEQ ID NOs:1 and 27, respectively. The cDNA and deduced amino acid residue sequence for cynomolgus monkey CETP have also been reported by Pape et al., Atherosclerosis and Thrombosis, 11:1759-1771 (1991), and that polypeptide of SEQ ID NO:30 or a portion thereof as described below, can also be utilized herein, as can the cDNA of that monkey shown in SEQ ID NO:31 or a portion thereof.

Where the whole CETP molecule is used alone as the immunogenic polypeptide of the CETP immunogen, it is preferred to use a recombinant protein as compared to using protein recovered from an animal. It is also preferred to use a protein from an animal species other than that of the immunized mammal; i.e., the CETP used is preferably xenogeneic. An example of the use of a recombinant CETP protein from one animal in an immunized host mammal of another species is illustrated hereinafter by the use of recombinant human CETP in rabbits.

In an alternative procedure, a CETP protein is reacted with a plurality of diazotized sulfanilic acid molecules to form a modified CETP protein that itself serves as the CETP immunogen as is disclosed in U.S. Pat. No. 4,767,842 for the human luteinizing hormone (HLH), whose disclosures are incorporated herein by reference. When this type of modification is utilized, the CETP protein used is preferably from the same species (allogeneic) as the immunized mammal.

When an immunogenic polypeptide is other than an intact CETP molecule, it is preferred to use a polypeptide having a length of about 10 to about 30 amino acid residues, and more preferably, a length of about 20 to 30 residues. In this instance, the immunogenic polypeptide is covalently bonded to an exogenous antigenic carrier to form the CETP immunogen. Several means are known in the art for covalently bonding polypeptides together, and several such means are discussed hereinafter. It is preferred that the covalent bond used to link the exogenous antigenic carrier and immunogenic polypeptide be a peptide bond. Several methods for forming peptide bonds are also well known in the art, but the preferred method of forming that bond is by expression of a fusion protein.

Exogenous antigenic carrier polypeptide molecules are also well known in the art, as are the amino acid residue and nucleotide sequences of those molecules. Exemplary polypeptide carriers include but are not limited to tetanus toxoid, tuberculin purified protein derivative (PPD), diphtheria toxoid, thyroglobulin and the hepatitis B core protein (HBcAg).

Thus, the cDNA encoding an exogenous antigenic carrier and that encoding an immunogenic CETP polypeptide can be operatively linked to form a single isolated and purified DNA molecule that encodes both the carrier and immunogenic polypeptide. That DNA molecule can then be operatively linked in an appropriate expression vector that expresses those two polypeptides as a single fusion protein whose two polypeptide portions are covalently bonded by a peptide bond. Preferably, the carrier is expressed at the amino-terminus of the fusion protein, although a carrier can also be expressed at the carboxy-fusion terminus of the immunogenic polypeptide. Exemplary proteins and procedures for their synthesis are discussed hereinafter.

Preferably, where the whole CETP molecule is used as the immunogenic polypeptide, the carrier polypeptide has an amino acid residue sequence that is less than that of a whole protein. That length is preferably about 15 to about 70 amino acid residues.

The hepatitis B nucleocapsid or core protein antigen also referred to as HBcAg is a particularly preferred exogenous antigenic carrier, as will be discussed in greater detail hereinafter. The HBcAg molecule will often be used herein illustratively as a carrier.

U.S. Pat. No. 4,818,527, whose disclosures are incorporated by reference, teaches that the region extending from about position 70 through about position 140 from the amino-terminus of HBcAg, whose complete amino acid and cDNA sequences are shown as SEQ ID NOs:38 and 39, respectively, is particularly useful as a T cell independent stimulant as are sequences of about 15 to about 25 residues from that region. The amino acid residue sequences of four of those shorter polypeptides are provided as SEQ ID NOs:40, 41, 42 and 43. The cDNA sequences that encode each of those four polypeptides can be readily obtained from SEQ ID NO:39, and the 3′ end of such a cDNA can be operatively linked to the 5′ end of cDNA that encodes an immunogenic polypeptide, or vice versa, for expression as a fusion protein CETP immunogen.

Thus, in one embodiment, a preferred CETP immunogen is a fusion protein whose amino-terminal portion is a polypeptide having a length of about 15 to about 70 amino acid residues and having the sequence of HBcAg from about position 70 to about position 140 from the HBcAg amino-terminus. The carboxy-terminal portion of that fusion protein has the amino acid residue sequence of a CETP molecule, and the two portions are covalently bonded by a peptide bond. In this embodiment, the CETP molecule can be from the same species as the immunized mammal.

In another preferred embodiment, the CETP immunogen is comprised of an exogenous antigenic carrier to which one or more immunogenic polypeptides having a length of about 10 to about 30 amino acid residues such as those of SEQ ID NOs:2-7 or 50 having a sequence of rabbit CETP, the similar polypeptides of SEQ ID NOs:8-13 or 29 having a sequence of human CETP or the similar polypeptides of SEQ ID NOs:32-37 having a sequence of monkey CETP is covalently bonded. Here, the carrier is preferably an intact protein such as a before-noted tetanus toxoid, tuberculin PPD, diphtheria toxoid, thyroglobulin or HBcAg molecule or a synthetic carrier such as the branched oligolysine described in Tam et al., Proc. Natl. Acad. Sci., USA, 86:9084-9088 (1989) or the similarly prepared branched oligolysine that is also linked to resin particles as described in Butz et al., Pep Res. 7(1):20-23.

Methods for covalent bonding of an immunogenic polypeptide are extremely varied and are well known by workers skilled in the immunological arts. For example, following U.S. Pat. No. 4,818,527, whose disclosures are incorporated hereinby reference, m-maleimidobenzoyl-N-hydoxysuccinimide ester (ICN Biochemicals, Inc.) or succinimidyl 4-(N-maleimidomethyl)cyclohexane-1-carboxylate (SMCC, Pierce) is reacted with HBcAg to form an activated carrier. That activated carrier is then reacted with a polypeptide of SEQ ID NOs:2-7, 8-13, 29, 32-37 or 50 to which an additional amino- or carboxy-terminal cysteine residue has been added to form a covalently bonded CETP immunogen as a conjugate. Alternatively, the amino group of an immunogenic polypeptide can be first reacted with N-succinimidyl 3-(2-pyridylthio)propionate (SPDP, Pharmacia), and that thiol-containing polypeptide can be reacted with the activated carrier after reduction. Of course, the sulfur-containing moiety and double bond-containing Michael acceptor can be reversed. These reactions are described in the supplier's literature, and also in Kitagawa et al., J. Biochem., 79:233 (1976) and in P. J. Lachmann et al., in 1986 Synthetic Peptides As Antigens, Wiley, Chichester, (Ciba Foundation Symposium 119) pages 25-40.

Previously discussed U.S. Pat. No. 4,767,842 also teaches several modes of covalent attachment between a carrier and polypeptide that are useful here. In one method, tolylene diisocyanate is reacted with the carrier in a dioxane-buffer solvent at zero degrees C to form an activated carrier. An immunogenic polypeptide such as those of SEQ ID NOs:2-7, 8-13, 29, 32-37 or 50 is thereafter admixed and reacted with the activated carrier to form the CETP immunogen as a covalently bonded conjugate.

An exemplary antigenic carrier protein is the purified protein derivative (PPD) of tuberculin. Exemplary use of this carrier is discussed in P. J. Lachmann et al., in 1986 Synthetic Peptides As Antigens, Wiley, Chichester (Ciba Foundation Symposium 119) pages 25-40. Briefly, PPD is prepared from culture supernatants of Mycobacterium tuberculosis by ultrafiltration, heating to 100° C. and precipitation with trichloroacetic acid, and is available from commercial sources. This carrier is particularly useful for immunizing mammalian host animals that have been primed with Bacillus Calmatle-Guerin (BCG), as are humans who have been immunized against the tubercle bacillus.

In an exemplary coupling, PPD is thiolated using SPDP as described by the reagent's manufacturer. This technique can provide up to about five thiol-groups per PPD molecule. An immunogenic polypeptide such as those of SEQ ID NOs:2-7, 8-13, 29, 32-37 or 50 is coupled via its amino-terminal amine to SMCC, and after reduction of the thiolated PPD, the thiolated PPD and SMCC-reacted peptide are reacted as described by the reagent manufacturers to form a CETP immunogen as a covalently bonded conjugate.

An immunogenic polypeptide having a length of about 10 to about 30 amino acid residues, when desired as a free molecule, is usually most easily made by solid phase synthesis techniques, as are well known. Several such techniques are noted or cited in U.S. Pat. No. 5,582,997, whose disclosures are incorporated herein by reference.

Where a longer immunogenic polypeptide is desired, or where the immunogenic polypeptide is a portion of a CETP immunogen that is a fusion protein, it is preferred to utilize recombinant DNA synthetic techniques. DNA sequences for the CETP molecule or a desired portion thereof can be obtained as described by M. E. Pape et al., Arteriosclerosis and Thrombosis, 11:1759-1771 (1991); N. W. Jeong et al., Mol. Cells, 4(4):529-533 (1994); and D. T. Connolly et al., Biochem. J., 320:39-47 (1996). Oligonucleotides can also be prepared using standard synthetic technology where shorter DNA sequences are desired. Those oligonucleotides can also be linked enzymatically, as with T4 DNA ligase, to form longer molecules.

DNA sequences for exogenous antigenic carrier molecules have also been reported as have methods for expressing those molecules. For example, a DNA sequence that encodes the preferred HBcAg exogenous antigenic carrier is disclosed in U.S. Pat. No. 4,710,463, whose disclosures are incorporated herein by reference, and E. coli-containing plasmids whose DNA encode hepatitis B virus proteins were deposited in the Culture Collection of the National Collection of Industrial Bacteria, Aberdeen Scotland as pBR322-HBV G-L. In addition, DNA encoding HBcAg is disclosed in U.S. Pat. No. 4,942,125 as present in vectors deposited at the American Type Culture Collection (ATCC), 12301 Parklawn Drive, Rockville, Md. 20852-1776 as ATCC No. 39629, No. 39631 and No. 40102.

The use of HBcAg as an exogenous antigenic carrier in a fusion protein is illustrated in Moriarty et al., Vaccines 90, Brown et al. eds., Cold Spring Harbor Laboratory Press, Cold Spring Harbor, N.Y., 225-229 (1990). The authors there reported operatively linking the 3′ end of DNA encoding a 17-mer amino acid residue sequence of the HIV gag protein to the 5′ end of DNA encoding HBcAg, and reported that appropriately transfected E. coli expressed a fusion protein having the HIV gag sequence peptide-bonded to the amino-terminus of HBcAg. That expressed fusion protein was present in particulate form and was shown to be an excellent immunogen in mice.

Schödel et al., Vaccines 90, Brown et al. eds., Cold Spring Harbor Laboratory Press, 193-198 Cold Spring Harbor, N.Y. (1990) reported the preparation and successful use of a fusion protein immunogen that contained a polypeptide immunogen having an amino acid residue sequence of hepatitis B Pre-S2 (residues 133-140) that was expressed peptide-bonded to the carboxy-terminus of HBcAg so that the 3′ end of the exogenous carrier (HBcAg) DNA was linked to the 5′ end of the DNA that encoded the Pre-S2 polypeptide immunogen. That expressed fusion protein immunogen was also obtained in particulate form.

Similar techniques can be utilized here using a DNA molecule of SEQ ID NOs:14-19, 20-25 or a corresponding DNA sequence of SEQ ID NO:31 that encodes a CETP immunogenic polypeptide in place of the DNAs used by the Moriarty et al. or the Schödel et al. groups.

In addition, using similar techniques and others well known to workers of ordinary skill in the recombinant DNA art, a fusion protein can be prepared having an HBcAg amino acid residue sequence such as one of those of SEQ ID NOs:40-43 peptide-bonded to the amino-terminus of an intact CETP molecule.

A particularly preferred CETP immunogen is a fusion protein comprised of an immunogenic polypeptide having a length of 10 to about 30 amino acid residues that is peptide bonded to both an amino-terminal flanking amino acid residue sequence and a carboxy-terminal flanking sequence, and is sometimes referred to hereinafter HBcAg/CETP/HBcAg. Those flanking sequences are preferably portions from the amino-terminal and carboxy-terminal regions of the HBcAg molecule, as was discussed previously. Thus, in this fusion protein, the exogenous antigenic carrier molecule is peptide-bonded to both the amino-terminus and carboxy-terminus of the immunogenic polypeptide.

A preferred polypeptide immunogen has an amino acid residue sequence of SEQ ID NOs:2-7, 8-13, 29, 32-37 or 50. Most preferably, the polypeptide immunogen has an amino acid residue sequence that is bound by (immunoreacts with) the monoclonal antibodies designated TP1, TP2 and TP3 reported by B. Hessler et al., J. Biol. Chem., 263(11):5020-5023 (1988), or that denominated 1C4 by J. Gaynor et al., Atherosclerosis, 110(1):101-109 (1994). Monoclonal antibody TP2 binds to an epitope located between about positions 465 and 475 of human CETP. Tall, J. Lipid Res., 34:1255-1274 (1993), and the citations therein.

A particularly preferred polypeptide immunogen has an amino acid residue sequence that includes positions 465 through 475 of human CETP or an analogous position of CETP from another source, and is exemplified by the polypeptides of SEQ ID NOs:4, 10 and 34, as well as SEQ ID NO:29, of which the polypeptides of SEQ ID NOs:10 and 29 are most preferred, with SEQ ID NO:10 being encoded by the DNA of SEQ ID NO:22.

Protein molecules have not only a linear amino acid residue or primary sequence, but also can possess a secondary sequence in which the polypeptide back bone is coiled in an α-helix or folded into a β-sheet, as well as a tertiary sequence in which sequentially distant portions of the molecule are folded to be adjacent to each other. Many linear antigenic/immunogenic polypeptide sequences have been reported in the literature, and such sequences can be readily mimicked by polypeptides having a length of 10 to about 30 amino acid residues. Such relatively short polypeptides typically do not mimic a secondary structure such as an α-helix in aqueous media.

The region of CETP that immunoreacts with monoclonal antibody TP2 is predicted to have an amphipathic helical secondary structure, with the hydrophilic surface bound by the antibody. See Wang et al., J. Biol. Chem., 267(25):17487-17490 (1992) and A. R. Tall, J. Lipid Res. 34:1255-1274 (1993). A contemplated CETP immunogen fusion protein having an immunogenic polypeptide flanked at its amino- and carboxy-termini by peptide-bonded regions of HBcAg; i.e., HBcAg/CETP/HBcAg, is more constrained in its molecular motions than is an immunogenic polypeptide that is bonded at only one terminus. As a consequence, by flanking a before-mentioned particularly preferred immunogenic polypeptide with regions of HBcAg to form a HBcAg/CETP/HBcAg fusion protein, it is believed that the immunogenic polypeptide becomes constrained to have a helical structure much like that present in the native CETP molecule and thereby induce autogeneic antibodies having an antigenic specificity similar to those exhibited by mouse monoclonal antibodies TP1, TP2, TP3 and 1C4 discussed previously.

It is further believed that formation of HBcAg-like particles of a contemplated fusion protein HBcAg/CETP/HBcAg immunogen places further conformational constraints upon the immunogenic polypeptide by which the immunogenic polypeptide becomes the primary immunogen with loss of much of the HBcAg immunogenicity, while the T cell-independent antigenic carrier function of HBcAg is retained. See Schödel et al., J. Virol., 66(l):106-114 (1992) for a similar result using a different immunogen.

Although use of the full length HBcAg exogenous antigenic carrier molecule or substantially full length molecule has thus far been discussed, it is noted that about 10 amino-terminal amino acid residues or about 40 carboxy-terminal amino acid residues can be deleted from the expressed HBcAg/CETP/HBcAg sequence without abrogating function as an exogenous antigenic carrier or assembly into particles. See, for example, Birnbaum et al., J. Virol., 64(7):3319-3330 (1990).

Exemplary preparations of immunogenic fusion proteins having HBcAg as a carrier with various heterologous polypeptide insertions from pathogens as immunogen, and also usage of full length and carboxy-terminal deletions in the HBcAg amino acid residue sequence can be found in the following publications. Schödel et al., J. Exp. Med., 180:1037-1046 (1994); Schödel et al., J. Virol., 66(1):106-114 (1992); Schödel et al., Vaccines 91, Brown et al. eds., Cold Spring Harbor Laboratory Press, Cold Spring Harbor, N.Y., 319-325 (1991); Clarke et al., Vaccines 91, Brown et al. eds, Cold Spring Harbor Laboratory Press, Cold Spring Harbor, N.Y., 313-318 (1991); and Schödel et al., Vaccines 90, Brown et al. eds., Cold Spring Harbor Laboratory Press, Cold Spring Harbor, N.Y., 193-198 (1990).

It is also noted that the human hepatitis virus (HBV), whose core antigen is discussed herein, has two subtypes that are denominated adw and ayw. The core antigens of those two viral subtypes have slightly different DNA and amino acid residue sequences. Although subtype specificity has been noted as to the immunogenicity of the S and PreS regions of HBV, [see, for example, Milich et al. Vaccines 86, Brown et al. eds., Cold Spring Harbor Press, Cold Spring Harbor, N.Y., 377-382 (1986)] either subtype can be used as an exogenous antigenic carrier herein, with subtype ayw being used illustratively herein.

B. Inocula

A CETP immunogen is dissolved or dispersed in a pharmaceutically acceptable vehicle composition that is preferably aqueous to form an inoculum that, when administered to a mammal whose blood contains CETP in an effective amount, induces the production of antibodies that immunoreact with (bind to) CETP and lessen the transfer of cholesteryl esters from HDL particles.

Inocula typically contain CETP immunogen concentrations of about 10 micrograms to about 500 milligrams per inoculation (unit dose), and preferably about 50 micrograms to about 50 milligrams per unit dose. The term “unit dose” as it pertains to an inoculum of the present invention refers to physically discrete units suitable as unitary dosages for animals, each unit containing a predetermined quantity of active material calculated to individually or collectively produce the desired immunogenic effect in association with the required diluent; i.e., carrier, or vehicle.

Inocula are typically prepared from a dried solid CETP immunogen by dispersing the immunogen in a physiologically tolerable (acceptable) diluent vehicle such as water, saline phosphate-buffered saline (PBS), Ringer's solution or the like to form an aqueous composition. The diluent vehicle can also include oleaginous materials such as peanut oil, squalane or squalene as is discussed hereinafter. The amount of CETP immunogen utilized in each immunization can vary widely, and is referred to as an effective amount. Such an effective amount is sufficient to induce antibodies to CETP that bind to CETP and lessen the transfer of cholesteryl esters from HDL particles. Exemplary effective amounts of CETP immunogen are about 500 μg to about 500 mg, depending inter alia, upon the CETP immunogen, mammal immunized, and the presence of an adjuvant in the inoculum, as discussed below. Thus, a single unit dose or a plurality of unit doses can be used to provide an effective amount of CETP immunogen.

Inocula can also include an adjuvant as part of the diluent. Adjuvants such as complete Freund's adjuvant (CFA) that is not used in humans, incomplete Freund's adjuvant (IFA) and alum are materials well known in the art, and are available commercially from several sources. The use of small molecule adjuvants is also contemplated herein.

Exemplary of one group of small molecule adjuvants are the so-called muramyl dipeptide analogues described in U.S. Pat. No. 4,767,842. Another type of small molecule adjuvant described in U.S. Pat. No. 4,787,482 that is also useful herein is a 4:1 by volume mixture of squalene or squalane and Arlacel™ A (mannide monooleate).

Yet another type of small molecule adjuvant useful herein is a 7-substituted-8-oxo or sulfo-guanosine derivative described in U.S. Pat. Nos. 4,539,205, 4,643,992, 5,011,828 and 5,093,318, whose disclosures are incorporated by reference. Of these materials, 7-allyl-8-oxoguanosine (loxoribine) is particularly preferred. That molecule has been shown to be particularly effective in inducing an antigen-(immunogen-)specific response.

Adjuvants are utilized in an adjuvant amount, which can vary with the adjuvant, mammal and CETP immunogen. Typical amounts can vary from about 100 μg to about 200 mg per immunization. Those skilled in the art know that appropriate concentrations or amounts can be readily determined.

An inoculum is typically formulated for parenteral administration. Exemplary immunizations are carried out sub-cutaneously (s.c.) intra-muscularly (i.m.) or intra-dermally (i.d.).

Once immunized, the mammal is maintained for a period of time sufficient for the CETP immunogen to induce the production of antibodies that bind to CETP and lessen the transfer of cholesteryl esters from HDL particles. This maintenance time typically lasts for a period of about three to about eight weeks, and can include a booster, second immunizing administration of the inoculum.

The production of antibodies that bind to CETP is readily ascertained by obtaining a plasma or serum sample from the immunized mammal and assaying the antibodies therein for their ability to bind to CETP as an antigen in an ELISA assay as described hereinafter, or by another immunoassay such as a Western blot as is well known in the art.

The lessening of transfer of cholesteryl esters from HDL can be assayed by one or more of several techniques. In one assay, the rate of transfer is measured by use of a [³H]-cholesteryl ester ([³H]CE) from HDL to LDL following the differential precipitation assay reported by Glenn et al., Methods in Enzymology, 263:339-350 (1996). Briefly, in a volume of 200 μl, CETP, [³H]CE-labeled HDL, LDL, and TES assay buffer (50 mM Tris, pH 7.4; 150 mM NaCl; 2 mM EDTA; 1% bovine serum albumin) are incubated for 2 hours at 37° C. in 96-well filter plates. LDL is then differentially precipitated by the addition of 50 μl of 1% (w/v) dextran sulfate/0.5 M MgCl₂. After filtration, the radioactivity present in the precipitated LDL is measured by liquid scintillation counting. Correction for non-specific transfer or precipitation is made by including samples that did not contain CETP. The rate of [³H]CE transfer using this assay is linear with respect to time and CETP concentration. For studies in which antibodies are included in the assay, the order of addition into sample wells is: buffer, [³H]CE-labeled HDL, LDL, antibodies, CETP.

CETP activity can also be measured using two methods that do not involve differential precipitation. In the first assay, the incubation conditions are identical to those described above, but separation of LDL acceptor particles from [³H]CE-labeled HDL donor particles is accomplished by size exclusion chromatography on tandem columns of Superose™ 6 (Sigma Chemical Co.), followed by liquid scintillation counting of fractions to determine the amount of [³H]CE associated with LDL and HDL. The amount of transfer measured by this method is typically in excellent agreement with the precipitation assay.

The third assay for CETP activity measures the rate of CETP-mediated transfer of the fluorescent analog NBD-cholesteryl linoleate (NBD-CE) from an egg phosphatidyl choline emulsion to VLDL. This assay takes advantage of the fact that NBD-CE is self-quenched when in the emulsion, and becomes fluorescent when transferred to VLDL. The assay is carried out according to the manufacturer's instructions (Diagnescent Technologies Inc., Yonkers, N.Y.). Fluorescence measurements can be taken using a standard machine such as an SLM 8000C spectrophotofluorometer (Milton Roy Co., Rochester, N.Y.) using 465 nm and 535 nm for excitation and emission wavelengths, respectively.

It is particularly contemplated once the desired antibodies are induced in the mammal that the immunization step be repeated at intervals of about 3 to about 6 months until the HDL cholesterol value in the blood of the mammal is increased by about 10 percent or more relative to the HDL cholesterol value for the mammal prior to the first immunization step. Preferably, the HDL cholesterol value is increased by about 25 percent. The mammal is thereafter preferably maintained at that increased HDL cholesterol level by periodic booster immunizations administered at intervals of about 6 to about 18 months. The increase in HDL cholesterol can be measured by any reliable assay, many of which are well known in the art, and one of which is described hereinafter.

It is noted that the before-described anti-CETP antibodies so induced can be isolated from the blood of the host mammal using well known techniques, and then reconstituted into a second inoculum for passive immunization as is also well known. Similar techniques are used for gamma-globulin immunizations of humans. For example, antiserum from one or a number of immunized hosts can be precipitated in aqueous ammonium sulfate (typically at 40-50 percent of saturation), and the precipitated antibodies purified chromatographically as by use of affinity chromatography in which CETP or an immunogenic polypeptide portion thereof is utilized as the antigen immobilized on the chromatographic column.

C. DNA Molecules and Expression Systems

A contemplated DNA molecule (isolated purified DNA segment) that encodes a CETP immunogen can be referred to as a number of base pairs at a particular location in a plasmid, as a restriction fragment bounded by two restriction endonuclease sites, and as a restriction fragment bounded by two restriction endonuclease sites and containing a number of base pairs. A contemplated DNA can also be defined to have a sequence of a denominated SEQ ID NO, as well as alleles or variants of such genes (described hereinafter) that encode a recited amino acid residue sequence.

A contemplated isolated and purified DNA segment is linear, and as such has a 5′ end and a 3′ end. A contemplated DNA segment can comprise two or more individual DNA segments whose 3′ ends are operatively linked to the 5′ end of another DNA segment where two segments are joined, or whose 3′ end is operatively linked to the 5′ end of another DNA segment whose own 3′ end is operatively linked to the 5′ end of yet another DNA segment, where three individual segments are joined to form a single isolated and purified DNA segment.

In living organisms, the amino acid residue sequence of a protein or polypeptide is directly related via the genetic code to the deoxyribonucleic acid (DNA) sequence of the structural gene that codes for the protein. A structural gene can be defined in terms of the amino acid residue sequence; i.e., protein or polypeptide, for which it codes.

In addition, through the well-known redundancy of the genetic code, additional DNA sequences can be prepared that encode the same amino acid residue sequences, but are different from a recited gene sequence having a particular SEQ ID NO. For example, in vitro mutagenesis as is illustrated hereinafter can be used to change a DNA sequence so that the same residue of an expressed polypeptide is expressed using one or more different codons. In addition, that same technique can be used to change one amino acid residue to another where it is desired to insert or delete specific restriction endonuclease sites. This technique is also illustrated hereinafter.

A DNA sequence that encodes a CETP immunogen of a recited SEQ ID NO but has a DNA sequence different from that of a recited SEQ ID NO is referred to herein as a variant DNA sequence. Such a variant DNA molecule can be readily prepared by in vitro mutagenesis, as is well known.

A DNA segment that encodes a described CETP immunogen can be synthesized by chemical techniques, for example, the phosphotriester method of Matteucci et al., J. Am. Chem. Soc., 103:3185 (1981). Of course, by chemically synthesizing the coding sequence, any desired modifications can be made simply by substituting the appropriate bases for those encoding the native amino acid residue sequence.

However, DNA segments including the specific sequences discussed previously are preferred. Furthermore, a DNA segment that encodes a polypeptide can be obtained from a recombinant DNA molecule (plasmid or other vectors) containing that segment.

A DNA segment that includes a DNA sequence encoding a CETP immunogen can be prepared by excising and operatively linking appropriate restriction fragments from appropriate plasmids or other DNA using well known methods. The DNA molecules useful here that are produced in this manner typically have cohesive termini; i.e., “overhanging” single-stranded portions that extend beyond the double-stranded portion of the molecule. The presence of cohesive termini on the DNA molecules of the present invention is preferred, although molecules having blunt termini are also contemplated.

A recombinant DNA molecule useful herein can be produced by operatively linking a vector to an isolated DNA segment that encodes a CETP immunogen to form a plasmid such as those discussed herein. Particularly preferred recombinant DNA molecules are discussed in detail in the examples, hereafter. Vectors capable of directing the expression of the gene are referred to herein as “expression vectors”.

The expression vectors described above contain expression control elements including a promoter. The genes that encode an immunogenic polypeptide or other useful sequence are operatively linked to the expression vector to permit the promoter sequence to direct RNA polymerase binding and expression of the desired polypeptide coding gene. Useful promoters for expressing the polypeptide coding gene are inducible.

The choice of which expression vector to which a polypeptide-coding gene is operatively linked depends directly on the functional properties desired, e.g. the location and timing of protein expression, and the host cell to be transformed. These are well known limitations inherent in the art of constructing recombinant DNA molecules. However, a vector useful in practicing the present invention is capable of directing the replication and also the expression of the immunogenic polypeptide-coding gene included in the DNA segment to which it is operatively linked.

In one preferred embodiment, a vector includes a prokaryotic replicon; i.e., a DNA sequence having the ability to direct autonomous replication and maintenance of the recombinant DNA molecule extrachromosomally in a prokaryotic host cell transformed therewith. Such replicons are well known in the art.

Those vectors that include a prokaryotic replicon can also include a prokaryotic promoter region capable of directing the expression of the CETP immunogen gene in a host cell, such as E. coli, transformed therewith. Promoter sequences compatible with bacterial hosts are typically provided in plasmid vectors containing one or more convenient restriction sites for insertion of a DNA segment of the present invention. Typical of such vector plasmids are pUC18, pUC19, and pBR322 available from Gibco BRL, Gaithersburg, Md., and pPL and pKK223-3 available from Pharmacia, Piscataway, N.J. These vectors are utilized in the synthesis of the DNA segments useful herein.

In preferred embodiments, the vector used to express an immunogenic polypeptide-coding gene includes a selection marker that is effective in a host cell, preferably a drug resistance selection marker. One preferred drug resistance marker is the gene whose expression results in kanamycin resistance, whereas ampicillin resistance is another such marker. Again, such selective markers are well known.

A variety of methods has been developed to operatively link DNA to vectors via complementary cohesive termini or blunt ends. For instance, complementary homopolymer tracts can be added to the DNA segment to be inserted and to the vector DNA. The vector and DNA segment are then joined by hydrogen bonding between the complementary homopolymeric tails to form recombinant DNA molecules.

Alternatively, synthetic linkers or adapters containing one or more restriction endonuclease sites can be used to join the DNA segment to the integrating expression vector. The synthetic linkers or adapters are attached to blunt-ended DNA segments by incubating the blunt-ended DNA segments with a large excess of synthetic linker or adapter molecules in the presence of an enzyme that is able to catalyze the ligation of blunt-ended DNA molecules such as bacteriophage T4 DNA ligase.

Thus, the products of the reaction are DNA segments carrying synthetic linker sequences at their ends. These DNA segments are then cleaved with the appropriate restriction endonuclease and ligated into an expression vector that has been cleaved with an enzyme that produces termini compatible with those of the synthetic linker. Synthetic linkers containing a variety of restriction endonuclease sites are commercially available from a number of sources including New England BioLabs, Beverly, Mass. A synthetic adapter molecule typically has sticky end and one blunt end and is not cleaved after ligation.

Although preferred, it is not always feasible to design a DNA molecule whose expressed polypeptide has the exact terminal residues of a polypeptide enumerated in a SEQ ID NO. This is because of the limitations inherent in the use of restriction enzymes, synthetic linkers and adapter molecules used for cutting and joining DNA segments.

As a consequence, an expressed polypeptide can contain a few (e.g. one or two) more, less or different amino acid residues at one or both termini of an enumerated sequence. Such slight changes are well tolerated by a contemplated CETP immunogen, particularly when the substitution is conservative and residues such as Cys and Pro are avoided.

### BEST MODE FOR CARRYING OUT THE INVENTION

### EXAMPLE 1

Immunization of Rabbits with Rabbit CETP-Peptides

There is a 88 percent homology between rabbit and human CETP at the amino acid residue level. Rabbits express high levels of CETP in their blood and were chosen as a model for illustrating production of autogeneic anti-CETP antibodies.

The six rabbit CETP polypeptides of SEQ ID NO:2-7 were selected for this study and were prepared by standard solid phase synthesis procedures discussed below. To enhance the anti-polypeptide-specific antibody responses, two separate immunization strategies were used with the above six rabbit CETP-polypeptides.

A. Immunization Strategy 1 (MAP Conjugates)

Rabbit polypeptides were synthesized as multiple antigenic peptide (MAP) constructs [D. N. Posnett et al., J. Biol Chem., 263:1719-1725 (1988)]. Those polypeptides were separately covalently bonded to “oligolysine core” molecules that were themselves covalently attached to resin particles [S. Butz et al., Pep. Res. 1:20-223 (1994)].

The substitution of the starting resin particles was 0.37 μm sites/mg resin that provided approximately 500 μg of immunogenic polypeptide per 1.1 mg resin. For the preparation of the CETP immunogen for immunization, 3.0 mg of dry resin were weighed out and hydrated in 1.3 ml sterile phosphate-buffered saline (PBS; pH 7.4) to which 1.3 ml Freund's complete adjuvant (CFA; Sigma Chemical Co., St. Louis, Mo., F-5881) were added as adjuvant. The CETP immunogen and adjuvant were emulsified by a female-female luer lock syringe adapter connected to two 3 ml syringes. Each final emulsion was divided into 1.0 ml aliquots for injection (1 ml/rabbit), with one immunogen used per rabbit. Pre-immune rabbit serum was collected before immunization and stored at −70° C. until immunoassay. On day 1, New Zealand white rabbits were separately immunized with respective immunogens by sub-cutaneous (s.c.) route on the back of the rabbit using 10 injection sites.

Three weeks later (on Day 22), the rabbits were boosted using similar procedures, but this time CETP immunogens were emulsified in Freund's incomplete adjuvant (IFA; Sigma). The resin-bonded CETP immunogen was weighed out as before and hydrated with sterile PBS the day before the booster immunization. The resulting CETP immunogen suspension was sonicated with a microtip at maximum setting for 5 minutes and left overnight (about 18 hours) at 4° C. Before mixing the hydrated CETP immunogen suspension with IFA, the suspension was warmed to room temperature just before the booster immunization, added to 1.5 ml IFA, and emulsified as described above to form an inoculum in which the CETP immunogen was dispersed. Rabbits were immunized each with 1 ml of emulsion in at least 10 injection sites s.c.

The first post-immune serum was collected 2 weeks after the second immunization from each animal. All the anti-sera samples were stored in −70° C. until ELISA was done.

Using this MAP strategy, polypeptides of SEQ ID NOs:2 and 7 were moderately immunogenic in rabbits and resulted in maximum autogeneic antibody titers of 1:1000 and 1:300, respectively. The titers represent the dilution of the sera that gave a half maximal absorbance on ELISA plates coated with the respective polypeptides. Sera were pooled from two rabbits, and the above titers represent the mean value. Only anti-sera to SEQ ID NO:7 cross-reacted with recombinant human CETP. The reactivity of these anti-sera with rabbit CETP is under evaluation using various immunological assays. Anti-polypeptide-specific IgG has been purified from the post-immune sera and its inhibitory property on human recombinant CETP is being assayed.

B. Immunization Strategy 2 (Purified Protein Derivative Conjugates)

Five of the above six rabbit CETP-polypeptide immunogens (SEQ ID NOs:2, 3, 4, 6 and 7) were coupled to tuberculin purified protein derivative (PPD) according to the teachings of P. J. Lachmann et al., in 1986 Synthetic Peptides As Antigens, (Ciba Foundation Symposium 119), 25-40 (1986) and P. Dawson et al., J. Bio. Chem., 264:16798-16803 (1989) to form a conjugate. The tuberculin PPD (Statens Serum Inst., Copenhagen, Denmark) was used as an exogenous antigenic carrier to enhance the immunogenicity of rabbit CETP-derived polypeptides. The polypeptide-PPD conjugate in PBS was emulsified with CFA as described for immunization strategy 1. One ml of 0.5 mg/ml polypeptide conjugated to PPD was emulsified with approximately 1 ml CFA. A second 1 ml PPD-conjugate was frozen for next booster immunization.

On Day 1, rabbits were immunized with 1 ml of final emulsion in at least 10 sites sub-contaneously on back of the rabbit. The polypeptide-PPD CETP polypeptide immunogen dose contained 0.25 mg of polypeptide per rabbit. Three weeks later (on Day 21), the rabbits were given the booster immunization dose with the remaining 1 ml conjugate thawed and emulsified with IFA, as discussed before. Two weeks following the second immunization rabbits were bled to collect post-immune sera.

The PPD conjugation strategy resulted in antibodies to the immunogenic polypeptides of SEQ ID NOs:2 and 6, with antibody titers of 1:3200 and 1:400 respectively. The titers represent the dilution of the sera that gave a half maximal absorbance on peptide coated ELISA plates. Sera were pooled from two rabbits and represent the mean value. Only the antibodies to the immunogenic polypeptide of SEQ ID NO:2 cross-reacted with recombinant human CETP. These results were unexpectedly good inasmuch as P. J. Lachmann et al., supra, obtained substantially no anti-polypeptide antibodies in BCG-naive hosts as were these rabbits. Anti-PPD antibodies were detected in all groups of rabbits as expected.

Using ELISA, the anti-immunogenic polypeptide sera are being used to evaluate their immuno-reactivity with natural rabbit CETP. Because the polypeptides of SEQ ID NOs:2, 6 and 7 were immunogenic and the two anti-polypeptide antibodies against SEQ ID NOs:2 and 7 immunologically cross-reacted with recombinant human CETP, the respective rabbits were further boosted with a third immunization dose either with the MAP constructs or PPD constructs emulsified with IFA.

### EXAMPLE 2

Immunization of Outbred Rabbits with CETP-Based Antigen

This study utilized 30 New Zealand white rabbits in three groups with 10 rabbits per group. Three immunogens were utilized in this study: (1) Recombinant human CETP, (2) the carboxy-terminal 26 amino acid residues of rabbit CETP (SEQ ID NO:50), and (3) a control immunogen whose amino acid residue sequence was unrelated to that of CETP.

Pre-immune sera were collected before immunization with the respective immunogens. The purpose of this study was to illustrate that the above CETP immunogens would induce anti-CETP-specific (autogeneic anti-CETP) antibodies in rabbits, and that the autogeneic antibodies generated against CETP bind to (immunoreact with) the endogenous rabbit CETP, and thus lessen the transfer of cholesteryl esters from HDL particles and raise the level of HDL in the hosts.

The above immunogens were emulsified in CFA. Each rabbit received 500 μg of one of the immunogens emulsified in CFA immunized by sub-cutaneous route. Seven weeks later the first bleed post-immune sera were collected.

ELISA was employed to titrate the antibodies. ELISA plates were coated (40 ng/well) with the recombinant human CETP.

The rabbits immunized with recombinant human CETP exhibited a primary immune response against human CETP. All the ten rabbits responded well to the recombinant human CETP (rhCETP). The specific IgG antibody titer was >1:1000. However, the group of 10 rabbits immunized with the rabbit CETP carboxy-terminal polypeptide-thyroglobulin conjugate (CETP-TH) did not exhibit a primary antibody response. The control rabbit sera had no detectable levels of anti-CETP antibodies. The rabbits were boosted with each respective antigen to further study immunogenicity.

The results of this study on the elevation of HDL particle concentration in the blood (plasma) of the host mammals (mean ±.S.D.) are shown in Table 1, below, for those first-immune sera.

TABLE 1

| | HDL Levels In Immunized Animals (mg/dl) | | |
|---|---|---|---|
| | Avg.³ | | |
| Immunogen | HDL | S.D.⁴ | p⁵ |
| Control | 23.89 | 3.92 | — |
| rhCETP¹ | 26.59 | 4.41 | 0.17 |
| CETP-TH² | 26.14 | 6.93 | 0.38 |

¹rhCETP = Recombinant human CETP.
²CETP-TH = C-terminal 26 rabbit CETP amino acid residues conjugated to thyroglobulin.
³Avg. HDL = Average HDL concentration after immunization or mock immunization for the control.
⁴S.D. = Standard deviation.
⁵P = p value from a Student's T test analysis.

As can be seen from those results, an increase in HDL particle concentration was found after administration of each of the CETP immunogens. There was a relatively large scatter in the data. Nevertheless, an approximately 10 percent increase in the HDL particle level was observed with each CETP immunogen as compared with the control, with the recombinant human CETP immunogen providing its increase at a confidence level of greater than 80 percent (p=0.17) using a Student's T test to analyze the results.

### EXAMPLE 3

Construction of E. coli Expression Vectors Encoding HBcAg/CETP/HBcAg Chimeric Fusion Proteins

A. PCR Amplification of HBcAg

Plasmid pFS14, a derivative of expression vector pKK223 (Pharmacia), encodes HBcAg (subtype ayw) [Schödel et al., Infect. Immun. 57:1347 (1989)]. PCR primer A, below, is designed to amplify the 5′ end of HBcAg and place an NcoI (C′CATG,G) site in the correct reading frame at the natural ATG start codon. In each of the sequences shown hereinafter, only the coding strand is shown, and bases removed after cleavage by restriction enzymes are shown in lower case.

Primer A: 5′ gatccCATGGACATCGACCCTTATAAAGAATTTGG 3′ SEQ ID NO:44

Primer Z, below, is designed to amplify the 3′ end of HBcAg and place a TAA stop codon and a HindIII (A′AGCT,T) site following amino acid 183 (Cys).

Primer Z: 5′ gatcaAGCTTTTAACATTGAGATTCCCGAGATTGAGATCTTCTG 3′ SEQ ID NO:45

A DNA fragment encoding the full-length HBcAg with modified 5′ and 3′ ends is amplified using plasmid pFS14 DNA as a template in the presence of primer A and primer Z under the standard polymerase chain reaction conditions recommended by the manufacturer of the GeneAmp PCR reagent kit (Perkin Elmer Cetus, Norwalk, Conn.).

The amplified DNA is then cleaved with NcoI and HindIII, and fractionated by size on an agarose gel. Full-length HBcAg DNA is purified from a gel slice using a QIAQUICK™ gel extraction kit (QIAGEN, Chatsworth, Calif.).

B. Insertion Pf HBcAg Onto pProEx1

pProExl, an E. coli expression vector (Life Technologies, Inc., Gaithersburg, Md.), is also cleaved with NcoI and HindIII and gel-purified. The amplified DNA and pProEx1 DNAs are ligated under standard conditions using T4 DNA ligase and transformed into chemically-competent E. coli DH10B cells (Life Technologies, Inc.) using protocols supplied by the vendor to form plasmid ProEx1-AZ. The transformation mixture is spread on LB agar plates containing 100 μg/ml ampicillin and incubated overnight at (about 18 hours) at 37° C. Colonies harboring ampicillin-resistant plasmids are purified by restreaking on fresh LB agar plates containing ampicillin, and minipreps of plasmid DNA are prepared using WIZARD™ 373 DNA purification kits (Promega, Inc., Madison, Wis.). Plasmids containing the HBcAg fragment inserted into the NcoI and HindIII sites of pProEx1 are characterized by restriction mapping and sequence analysis across the inserted region.

Plasmid pProEx1-AZ is then modified to insert a polylinker between the nucleotides that encode amino acid residues 70-75 of HBcAg.

Primer B is designed to insert an XhoI site (C′TCGA) and an EcoRI site (G′AATT,C) site following position 206 of SEQ ID NO:39. Primer Y is designed to insert an EcoRI site (G′AATT,C) site followed by a SpeI site (A′CTAG,T) before position 226 of SEQ ID NO:39.

| | |
|---|---|
| Primer Y: 5′ gatcgAATTCACTAGTTGGAAGATCCAGCGTCTAGAGACCTAGTAG 3′ | SEQ ID NO:46 |
| Primer B: 5′ gatcgAATTCCTCGAGCTAGAGTCATTAGTTCCCCCCAGCA 3′ | SEQ ID NO:47 |

Plasmid pProEx1-AZ is then used as a template with primers A and B to amplify a segment of DNA (designated HBcAg-AB) encoding amino acid residues 1-69 of HBcAg to generate a fragment that contains an NcoI site at its 5′ end and an XhoI and a EcoRI site at its 3′ end. The same plasmid is also used with primers Y and Z to amplify a segment of DNA (designated HBcAg-YZ) encoding amino acid residues 76-183 of HBcAg to generate a fragment that contains EcoRI and SpeI sites at its 5′ end and a HindIII site at its 3′ end.

The PCR product from the reaction designed to produce plasmid HBcAg-AB is cleaved with NcoI and EcoRI and purified after agarose gel electrophoresis. The PCR product from a second reaction designed to produce plasmid HBcAg-YZ is cleaved with EcoRI and HindIII and purified after agarose gel electrophoresis. The two gel-purified fragments are then ligated in a triple ligation reaction to plasmid pProEx1 that had been treated with NcoI and HindIII and purified after agarose gel electrophoresis. The desired ligated plasmid, pProEx1-AB-YZ, is obtained by screening ampicillin-resistant colonies for plasmids that have the predicted structure by restriction analysis, and is confirmed by DNA sequencing across the whole HBcAg region, particularly the A, BY, and Z junctions.

C. Cloning of CETP Segment Encoding SEO ID NO:29

A stably transformed CHO cell line transfected with human CETP cDNA [Wang et al., J. Biol. Chem., 270:612-618 (1995); Wang et al., J. Biol. Chem., 267:17487-17490 (1992)] provides CETP cDNA that is used as a template to amplify a segment (nucleotides 1346 to 1431) of the CETP coding sequence (SEQ ID NO: 1) that encodes the human peptide (SEQ ID NO: 29; ArgAspGlyPheLeuLeuLeuGlnMetAspPheGlyPheProGluHisLeu LeuValAspPheLeuGlnSerLeuSer) that is bound by the antibody TP2; T. L. Swenson et al., J. Biol. Chem., 264:14318-14326 (1989).

Primer C, below, is designed to amplify a region from just upstream from the natural XhoI site at position 1346. Primer X, below, is designed to amplify a region at the 3′ end of the CETP gene, removing the TAG codon and replacing it with an Eco47III site (AGCIGCT) followed by an EcoRI site (G′AATT,C).

| | |
|---|---|
| Primer C: 5′ GATTATCACTCGAGATGGCTTCCTGCTGCTGCAG 3′ | SEQ ID NO:48 |
| Primer X: 5′ gatcgAATTCAGCGCTCAAGCTCTGGAGGAAATCCACCAG 3′ | SEQ ID NO:49 |

The CETP cDNA is then used as a template with primers C and X to amplify a segment of DNA (designated pCETP-CX) encoding amino acid residues 461-476 of CETP, that contains an XhoI site near its 5′ end and an Eco47III and EcoRI site at its 3′ end. This segment, CETP-CX, is then cleaved with XhoI and EcoR47III, and gel-purified. Plasmid pProEx1-AB-YZ is digested with SpeI and treated with T4 DNA polymerase to remove the 4-base 5′ overhangs and generate blunt ends. [See, J. Sambrook et al., Molecular Cloning, 2nd 3d., Cold Spring Harbor Press, Cold Spring Harbor N.Y., 8-23 (1989).]

The resulting plasmid is then treated with XhoI, gel-purified, and ligated to the segment CETP-CX that has an XhoI site at one end and a blunt end resulting from cleavage with Eco47III at the other end. The resulting plasmid, designated pProEx1-ABC-XYZ, is characterized by restriction analysis and by sequencing to confirm that it contains sequences encoding amino acid residues 461-476 of CETP replacing sequences that encoded amino acid residues 70-75 of HBcAg in the vector pProEx1-AZ.

### EXAMPLE 4

Expression of HBcAg/CETP/HBcAg Chimeric Fusion Proteins in E. coli

The pProEx1 vector is designed for the expression of foreign proteins in E. coli. This vector contains a gene conferring resistance to ampicillin and a pBR322 origin of replication for propagation in E. coli. It also has a multiple cloning site flanked by a 6 histidine sequence (6× His) and the recognition sequence for rTEV protease. This site allows for the removal of the 6× His tag from a fusion protein after purification. The vector also has a Trc promoter and lacI^{q} gene permitting inducible gene expression with isopropyl-β-D-thiogalactopyranoside (IPTG). A procaryotic ribosomal binding site is located upstream from the start of translation of the 6× His tag. A unique NcoI site is located at the first codon of the 6× His tag. Plasmids pProEx1 and a control plasmid, pProEx1-CAT, are obtained from Life Technologies, Inc.

E. coli DH10B strains individually harboring pProEx1, pProEx1-CAT, pProEx1-AZ, or pProEx1-ABC-XYZ are cultured overnight (about 18 hours), and used as inocula for cultures that are induced with IPTG under conditions recommended by the vendor. Cultures harboring plasmid pProEx1-AZ produce HBcAg and those harboring pProEx1-ABC-XYZ produce the desired HBcAg/CETP/HBcAg fusion protein as particles. These proteins lack the 6× His tag present in the original pProEx1 vector because the HBcAg sequences are inserted at the NcoI site at the beginning of the 6× tag. Cultures harboring pProEx1-CAT produce a protein that migrates on SDS-PAGE gels as expected for a His-tagged CAT fusion protein.

### EXAMPLE 5

Expression of HBcAg/CETP/HBcAg Chimeric Fusion Proteins in Baculovirus-Infected Insect Cells

Baculovirus-infected insect cells have been shown to express a wide variety of recombinant proteins (V. A. Luckow, Insect Cell Expression Technology, pp. 183-218, in Protein Engineering: Principles and Practice, J. L. Cleland et al. eds., Wiley-Liss, Inc, 1996). Heterologous genes placed under the control of the polyhedrin promoter of the Autographa californica nuclear polyhedrosis virus (AcNPV) are often expressed at high levels during the late stages of infection. In most cases, the recombinant proteins are appropriately processed and are functionally similar to their authentic counterparts.

Recombinant baculoviruses containing the chimeric HBcAg/CETP/HBcAg gene are constructed using the baculovirus shuttle vector system (Luckow et al., J. Virol., 67:4566-4579, 1993) sold commercially as the Bac-To-Bac™ baculovirus expression system (Life Technologies, Inc.).

Briefly, pProEx1-ABC-XYZ is digested with NcoI, treated with Klenow enzyme to fill in the ends, and digested with HindIII to release the entire fragment encoding the HBcAg/CETP/HBcAg fusion protein. This fragment is inserted into a baculovirus donor plasmid, pFastBac1, that is digested with BamHI, treated with Klenow enzyme, and digested with HindIII. The resulting plasmid has the sequences encoding the hybrid HBcAg/CETP/HBcAg gene inserted downstream from the polyhedrin promoter of AcNPV. The mini-Tn7 segment containing the polyhedrin/HBcAg/CETP/HBcAg expression cassette is then transposed to a baculovirus shuttle vector propagated in E. coli and colonies harboring composite (recombinant) vectors are identified by their color and an altered drug resistance patterns. Miniprep DNAs are prepared and transfected into cultured Spodoptera frugiperda (fall armyworm) Sf9 cells.

Stocks of recombinant viruses are prepared and expression of the recombinant protein is monitored by standard protocols (O'Reilly et al., Baculovirus Expression Vectors: A Laboratory Manual, W.H. Freeman and Company, New York, 1992; King, L. A., and Possee, R. D. The Baculovirus Expression System: A Laboratory Guide, Chapman & Hall, London, 1992).

### EXAMPLE 6

Expression of HBcAg/CETP/HBcAg Chimeric Fusion Proteins in Mammalian Cells

The HBcAg/CETP/HBcAg fusion protein is expressed in mammalian cell culture using the BHK/VP16 expression system (Hippenmeyer et al., Bio/Technology, 11:1037-1041, 1993). Briefly, the NcoI-HindIII fragment from plasmid pProEx1-ABC-XYZ is isolated by gel electrophoresis and purified as before. The fragment is treated with Klenow polymerase and all four nucleotide triphosphates to make the 5′ overhanging ends blunt.

The mammalian expression vector pMON3327 contains the SV40 polyadenylation signal sequence in the BamHI site of plasmid pUC18, and is used as the basis for further plasmid construction. Ligation of the IE175 promoter of herpes simplex virus (HSV-1) upstream of the SV40 polyadenylation signal sequence in vector pMON3327 provides mammalian expression vector pMON3360B. The IE175 promoter is responsive to the HSV-1 VP-16 transactivator.

Expression vector pMON3360B is digested with BamHI and the 5′ over hanging ends at the unique BamHI site are filled in using Klenow polymerase. The vector sequences and the HBcAg/CETP/HBcAg sequences are ligated overnight (about 18 hours) at 15° C. using T4 DNA ligase. The ligation mixture is transfected into competent E. coli and selected for ampicillin resistance. Plasmid DNA is isolated from the colonies and analyzed by restriction analysis for proper orientation of the HBcAg/CETP/HBcAg sequences in the pMON3360B vector. A plasmid with the correct orientation is designated pMON3360B-HBcAg-CETP. Plasmid pMON3360B-HBcAg-CETP is purified using Promega Maxiprep™ protocols from 400 ml cultures.

BHK/VP16 cells are plated at about 3×10⁵ cells per 60 mm culture dish 24 hours before transfection in growth medium consisting of DMEM/5% fetal bovine sera (Life Technologies). Ten micrograms of plasmid pMON3360B-HBcAg-CETP and 1 μg of plasmid pMON1118 are transfected into the cells using LipofectAmine™ (Life Technologies) as recommended by the manufacturer. Two days after tranfection, the cells are treated with trypsin/EDTA (Life Technologies) and plated in ten 100 mm dishes in growth medium containing hygromycin (Sigma). In about two weeks, surviving colonies are isolated using filter paper and expanded and assayed for expression of the HBcAg/CETP/HBcAg fusion protein.

The foregoing description and the examples are intended as illustrative and are not to be taken as limiting. Still other variations within the spirit and scope of this invention are possible and will readily present themselves to those skilled in the art.

1. A process for increasing the concentration of HDL cholesterol in the blood of a mammal whose blood contains cholesteryl ester transfer protein (CETP) that comprises the steps of:
(a) immunizing said mammal with an inoculum containing a CETP immunogen that is an immunogenic polypeptide having a CETP amino acid residue sequence and is dissolved or dispersed in a vehicle; and
(b) maintaining said immunized mammal for a time period sufficient for said immunogenic polypeptide to induce the production of antibodies that bind to CETP and lessen the transfer of cholesteryl esters from HDL.

2. The process according to claim 1 wherein said immunogenic polypeptide is recombinant human CETP.

3. The process according to claim 1 wherein said immunogenic polypeptide is covalently bonded to an exogenous antigenic carrier.

4. The process according to claim 3 wherein said exogenous antigenic carrier is thyroglobulin.

5. The process according to claim 1 wherein said immunizing step is repeated.

6. The process according to claim 1 wherein said immunogenic polypeptide has the amino acid residue sequence of rabbit CETP.

7. The process according to claim 1 wherein said exogenous antigenic carrier polypeptide is peptide-bonded to the amino-terminus of said immunogenic polypeptide.

8. The process according to claim 7 wherein said immunogenic polypeptide has the amino acid residue sequence of SEQ ID NOs:29 or 50.

9. A process for increasing the concentration of HDL cholesterol in the blood of a mammal whose blood contains cholesteryl ester transfer protein (CETP) that comprises the steps of:
(a) immunizing said mammal with an inoculum containing a vehicle in which is dissolved or dispersed a CETP immunogen that is (i) an exogenous antigenic carrier polypeptide that is peptide-bonded to (ii) an immunogenic polypeptide having a CETP amino acid residue sequence;
(b) maintaining said mammal for a time period sufficient for said immunogenic polypeptide to induce the production of antibodies that bind to CETP and lesson the transfer of cholesterol esters from HDL; and
(c) repeating said immunizing step until the HDL cholesterol value in the blood of said mammal is increased relative to the HDL cholesterol value prior to said first immunization step.

10. The process according to claim 9 wherein said immunogenic polypeptide has the amino acid residue sequence of SEQ ID NOs:29 or 50.

11. The process according to claim 10 wherein said exogenous antigenic carrier polypeptide is thyroglobulin that is peptide-bonded to the amino-terminus of said SEQ ID NO:50.

12. A cholesteryl ester transfer protein (CETP) immunogen that comprises an immunogenic polypeptide having a CETP amino acid residue sequence covalently bonded to an exogenous antigenic carrier.

13. The immunogen according to claim 12 wherein said immunogenic polypeptide has a length of about 10 to about 30 amino acid residues.

14. The immunogen according to claim 13 wherein said immunogenic polypeptide has the sequence of SEQ ID NOs:29 or 50.
